(19) United States
(12) Patent Application Publication
(10) Pub. No.: US 2019/0310248 A1
(43) Pub. Date: Oct. 10, 2019

(54) METHOD FOR REMOVING MICROORGANISM, CELL, TINY VESICLE SECRETED BY SAID MICROORGANISM OR SAID CELL OR VIRUS FROM CARRIER-IMMOBILIZED ANTIBODY

(71) Applicant: HORIBA, LTD., Kyoto-shi, Kyoto (JP)

(72) Inventors: Shiro MIYAKE, Kyoto-shi, Kyoto (JP); Tomomi YAMASAKI, Kyoto-shi, Kyoto (JP)

(21) Appl. No.: 16/062,509

(22) PCT Filed: Dec. 12, 2016
(86) PCT No.: PCT/JP2016/086946

§ 371 Date: Jun. 14, 2018

(30) Foreign Application Priority Data
Dec. 15, 2015 (JP) 2015-244569
Sep. 16, 2016 (JP) 2016-182211

(51) Int. Cl. G01N 33/543; G01N 33/569; G01N 33/574

(52) U.S. Cl. CPC G01N 33/54393; G01N 33/56916; G01N 33/56966; G01N 33/57446; G01N 33/56944

(57) ABSTRACT

The present invention provides a method for removing a microorganism, a cell, a vesicle secreted by the microorganism or the cell, or a virus from an antibody immobilized on a carrier and to which the microorganism or the like has been bound as an antigen. The present invention also provides an immunological detection method of the microorganism or the like, including removing the microorganism or the like from an antibody immobilized on a carrier and to which the microorganism or the like has been bound as an antigen.

### TECHNICAL FIELD

The present invention relates to a method for removing a microorganism, a cell, a vesicle secreted by the microorganism or the cell, or a virus from an antibody immobilized on a carrier, for use for an immunological detection method of the microorganism, the cell, the vesicle secreted by the microorganism or the cell, or the virus. Furthermore, the present invention relates to a method for immunologically detecting a microorganism, a cell, a vesicle secreted by the microorganism or the cell, or a virus, comprising removing a microorganism, a cell, a vesicle secreted by the microorganism or the cell, or a virus from an antibody immobilized on a carrier, contacting a microorganism, a cell, a vesicle secreted by the microorganism or the cell, or a virus as a new antigen with the carrier, and detecting the microorganism, the cell, the vesicle secreted by the microorganism or the cell, or the virus by an immunological method.

### BACKGROUND ART

Bacterial infection is regarded as an important disease even in the present day with good public health. For example, due to the provision and increase in the scale of the cold chain of food distribution, food poisoning mainly due to Enterohemorrhagic Escherichia coli (EHEC) often becomes a big problem. The causative bacterium of EHEC infection is Escherichia coli that produces Verotoxin (VT). In EHEC infection, various clinical symptoms are known from asymptomatic to fatal one. Particularly, hemolytic-uremic syndrome (HUS) that may occur following Enterohemorrhagic Escherichia coli infection is a serious disease that may cause death or sequelae such as renal function disorder, neurological disorder and the like. It is important to identify the object contaminated with EHEC in order to prevent the development and spread of EHEC infection.

As EHEC detected from patients and carriers, various serotypes are known, and O157 is the most common, and O26 and O111 are the second most common in Japan. In the detection of EHEC, it is a standard practice to set O-antigen commonly present on the surface of the outer membrane of EHEC as the detection target. Detection of EHEC using O-antigen can be carried out by an immunological detection method. The immunological detection method includes detection of an antigen by utilizing an antigen-antibody reaction. It is a detection method generally rapid, convenient and economical as well as superior in detection accuracy. In the immunological detection method, the antibody to be used may be a free antibody or an antibody immobilized on a carrier. To reuse the antibody and process a large amount of test samples at a low cost, an antibody immobilized on a carrier is preferably used. In this case, the antigen bound to the antibody immobilized on a carrier must be removed with ease from the antibody. By removing the antigen, the carrier on which the antibody was immobilized becomes ready for detecting antigen repeatedly. However, when a bacterium is to be detected as an antigen and the bacterium is detected using an antibody for trapping bacteria including EHEC, the binding between the bacterium and the antibody cannot be resolved thereafter. The carrier immobilized with the antibody used once for the method of detecting bacteria must be disposed, thus causing a problem of large economic burden (non-patent document 1).

Also in human cells, immunological detection methods are widely used at medical sites, basic medical laboratories and the like for classification of immunocytes, detection of cancer cells in tissues, and the like. In addition, it has recently become clear that exosomes secreted by cells play a major role in signal transduction between cells. To detect those exosomes, an immunological detection method for the surface protein thereof is also effective. However, in the detection of cells and exosomes, the binding between cells or exosomes and antibodies cannot be resolved after detection as in the case of bacteria, and the method could not be proposed. That is, the carrier used for detection need to be self-made and will be disposed, thus causing a problem of large economic burden (non-patent documents 2-4).

### DOCUMENT LIST

### Non-Patent Documents

non-patent document 1: Bulard, E.; Bouchet-Spinelli, A.; Chaud, P.; Roget, A.; Calemczuk, R.; Fort, S.; Livache, T. Carbohydrates as New Probes for the Identification of Closely Related Escherichia coli Strains Using Surface Plasmon Resonance Imaging. Anal. Chem. 2015, 87, 1804-1811.
non-patent document 2: Kato, K.; Ishimuro, T.; Arima, Y.; Hirata, I, Iwata, H. High-Throughput Immunophenotyping by Surface Plasmon Resonance Imaging. Anal. Chem. 2007, 79, 8616-8623.
non-patent document 3: L. M. Rupert, D.; Lasser, C.; Eldh, M.; Block, S.; P. Zhdanov, V.; O. Lotvall, J.; Bally, M.; Hook, F. Determination of Exosome Concentration in Solution Using Surface Plasmon Resonance Spectroscopy. Anal. Chem. 2014, 86, 5929-5936.
non-patent document 4: Yang, C.; Mejard, R.; J. Griesser, H.; O. Bagnaninchi, P.; Thierry, B. Cellular Micromotion Monitored by Long-Range Surface Plasmon Resonance with Optical Fluctuation Analysis. Anal. Chem. 2015, 87, 1456-1461.

### SUMMARY OF THE INVENTION

### Problems to be Solved by the Invention

The present invention aims to provide a method for removing a microorganism, a cell, a vesicle secreted by the microorganism or the cell, or a virus from an antibody immobilized on a carrier and to which the microorganism, the cell, the vesicle secreted by the microorganism or the cell, or the virus has been bound as an antigen. The present invention also aims to provide a method for immunologically detecting a microorganism, a cell, a vesicle secreted by the microorganism or the cell, or a virus, comprising removing a microorganism, a cell, a vesicle secreted by the microorganism or the cell, or a virus from an antibody immobilized on a carrier and to which a microorganism, a cell, a vesicle secreted by the microorganism or the cell, or a virus has been bound, contacting a microorganism, a cell, a vesicle secreted by the microorganism or the cell, or a virus as a new antigen with the carrier, and detecting the microorganism, the cell, the vesicle secreted by the microorganism or the cell, or the virus by an immunological method.

### Means of Solving the Problems

The present inventors have conducted intensive studies in an attempt to provide a method for removing a microorganism, a cell, a vesicle secreted by the microorganism or the cell, or a virus as an antigen from an antibody immobilized on a carrier and found that the microorganism, the cell, the vesicle secreted by the microorganism or the cell, or the virus can be removed from the antibody by flowing a gel on the surface of the carrier, which resulted in the completion of the present invention.

That is, the present invention provides

[1] a method for removing a microorganism, a cell, a vesicle secreted by the microorganism or the cell, or a virus from an antibody immobilized on a carrier and to which the microorganism, the cell, the vesicle secreted by the microorganism or the cell, or the virus has been bound as an antigen, the method comprising flowing a gel (primary gel) on a surface of the carrier;
[2] the method of [1], wherein the primary gel is a polysaccharide gel or a protein gel;
[3] the method of [2], wherein the polysaccharide is selected from the group consisting of agarose, agar and carrageenan;
[4] the method of [2], wherein the protein is gelatin;
[5] the method of any one of [1]-[4], wherein the primary gel has a breaking strength of 4-1100 g/cm²;
[6] the method of any one of [1]-[5], wherein the microorganism is Escherichia coli, Streptococcus pneumoniae or Pseudomonas aeruginosa, the cell is an animal cell, and the vesicle secreted by the microorganism or the cell is an exosome;
[7] the method of [1], further comprising washing after flowing and flowing again a gel (secondary gel) the same as or different from the primary gel, wherein the primary gel is a mixture further comprising a salt aqueous solution;
[8] the method of [7], wherein the primary gel and the secondary gel are each a polysaccharide gel or a protein gel;
[9] the method of [8], wherein the polysaccharide is selected from the group consisting of agarose, agar and carrageenan;
[10] the method of [8], wherein the protein is gelatin;
[11] the method of [7], wherein the primary gel is agarose gel and the secondary gel is gelatin gel;
[12] the method of any one of [7]-[11], wherein the primary gel and the secondary gel each have a breaking strength of 4-1100 g/cm²;
[13] the method of any one of [7]-[12], wherein the salt aqueous solution is an aqueous ammonium sulfate solution;
[14] the method of any one of [7]-[13], wherein the microorganism is Escherichia coli, Streptococcus pneumoniae or Pseudomonas aeruginosa, the cell is an animal cell, and the vesicle secreted by the microorganism or the cell is an exosome;
[15] a method for immunologically detecting a microorganism, a cell, a vesicle secreted by the microorganism or the cell, or a virus, comprising removing a microorganism, a cell, a vesicle secreted by the microorganism or the cell, a vesicle secreted by the microorganism or the cell, or a virus from an antibody immobilized on a carrier and to which the microorganism, the cell, the vesicle secreted by the microorganism or the cell, or the virus has been bound as an antigen, by flowing a gel (primary gel) on a surface of the carrier, contacting a test sample with the carrier, and detecting the microorganism, the cell, the vesicle secreted by the microorganism or the cell, or the virus by an immunological method;
[16] the method of [15], further comprising washing after flowing and flowing again a gel (secondary gel) the same as or different from the primary gel, wherein the primary gel is a mixture further comprising a salt aqueous solution;
[17] an apparatus for removing a microorganism, a cell, a vesicle secreted by the microorganism or the cell, or a virus from an antibody immobilized on a carrier and to which the microorganism, the cell, the vesicle secreted by the microorganism or the cell, or the virus has been bound as an antigen, the apparatus comprising a mechanism for flowing a gel on a surface of the carrier.

### Effect of the Invention

Using the method of the present invention for removing a microorganism, a cell, a vesicle secreted by the microorganism or the cell, or a virus as an antigen from an antibody, the antibody conventionally unsuitable for reuse can be reused.

### BRIEF DESCRIPTION OF THE DRAWINGS

FIG. 1 shows a Flow-cell equipped with a microarray type SPRi apparatus (Horiba, Ltd.: OpenPlex).

FIG. 2 shows a biochip (Horiba, Ltd.: CS-HD) dedicated to the microarray type SPRi apparatus (Horiba, Ltd.: OpenPlex). The shaded area indicates the portion where the antibody is immobilized. The hexagonal frame shows where the Gasket in FIG. 1 comes into contact.

FIG. 3 shows an amount of change in reflected light accompanying the SPR phenomenon induced by the binding of Escherichia coli to the surface of a sensor chip immobilized with an antibody against O-antigen of Escherichia coli (O111, O157). vertical axis: reflection rate (%), horizontal axis: time (sec)

FIG. 4 shows an amount of change in reflected light accompanying the SPR phenomenon induced by the binding of Streptococcus pneumoniae to the surface of a sensor chip immobilized with a capsular antigen of Streptococcus pneumoniae (35B, 15B/C). vertical axis: reflection rate (%), horizontal axis: time (sec)

FIG. 5 shows an amount of change in reflected light accompanying the SPR phenomenon induced by the binding of Escherichia coli to the surface of a sensor chip immobilized with an antibody against O-antigen of Escherichia coli (O26, O91). vertical axis: reflection rate (%), horizontal axis: time (sec)

FIG. 6 shows an amount of change in reflected light accompanying the SPR phenomenon induced by the binding of Escherichia coli to the surface of a sensor chip immobilized with an antibody against O-antigen of Escherichia coli (O103, O115). vertical axis: reflection rate (%), horizontal axis: time (sec)

FIG. 7 shows an amount of change in reflected light accompanying the SPR phenomenon induced by the binding of Escherichia coli to the surface of a sensor chip immobilized with an antibody against O-antigen of Escherichia coli (O121, O128). vertical axis: reflection rate (%), horizontal axis: time (sec)

FIG. 8 shows an amount of change in reflected light accompanying the SPR phenomenon induced by the binding of Escherichia coli to the surface of a sensor chip immobilized with an antibody against O-antigen of Escherichia coli (O145, O159). vertical axis: reflection rate (%), horizontal axis: time (sec)

FIG. 9 shows an amount of change in reflected light accompanying the SPR phenomenon induced by the binding of exosome to the surface of a sensor chip immobilized with an antibody against CD9 on HCT116-released exosome. vertical axis: reflection rate (%), horizontal axis: time (sec)

FIG. 10 shows an amount of change in reflected light accompanying the SPR phenomenon induced by the binding of mast cell to the surface of a sensor chip immobilized with an antibody against mouse mast cell CD117. vertical axis: reflection rate (%), horizontal axis: time (sec)

FIG. 11 shows an amount of change in reflected light accompanying the SPR phenomenon induced by the binding of Pseudomonas aeruginosa to the surface of a sensor chip immobilized with an antibody against Pseudomonas aeruginosa. vertical axis: reflection rate (%), horizontal axis: time (sec)

FIG. 12 shows an apparatus for removing an antigen, which includes a mechanism for flowing a gel on a surface of a carrier immobilized with an antibody to which the antigen is bound. inlet and outlet for gel/sample/buffer: test sample, wash buffer. supply port and exhaust port of gel, three-way valve and gel loop: gel supply control valve, supply control loop. flow cell: see FIG. 1.

### DESCRIPTION OF EMBODIMENTS

The present invention provides a method for removing a microorganism, a cell, a vesicle secreted by the microorganism or the cell (hereinafter sometimes to be simply indicated as “vesicle”), or a virus (hereinafter “a microorganism, a cell, a vesicle secreted by the microorganism or the cell, or a virus as an antigen” is also sometimes indicated as “microorganism or the like”) from an antibody immobilized on a carrier and to which the microorganism or the like has been bound as an antigen, the method comprising flowing a gel (hereinafter sometimes to be also indicated as primary gel of the present invention) on a surface of the carrier (hereinafter sometimes to be indicated as the removal method I of the present invention). Preferably, the antigen in the present invention is a microorganism, a cell or a vesicle secreted by the microorganism or the cell.

As a microorganism bound to an antibody in the removal method I of the present invention, eubacterium (hereinafter indicated simply as “bacterium”) and archaebacterium can be mentioned, of which bacterium is preferable. The bacterium is not particularly limited as long as it is a prokaryote having a cellular membrane constituted of fatty acid ester of glycerol 3-phosphate. It may be a gram-negative bacterium or gram-positive bacterium. Examples of the gram-negative bacterium include, but are not limited to, bacteria belonging to Neisseria, Branhamella, Haemophilus, Bordetella, Escherichia, Citrobacter, Salmonella, Shigella, Klebsiella, Enterobacter, Serratia, Hafnia, Proteus, Morganella, Providencia, Yersinia, Campylobacter, Vibrio, Aeromonas, Pseudomonas, Xanthomonas, Acinetobacter, Flavobacterium, Brucella, Legionella, Veillonella, Bacteroides, Fusobacterium and the like, of which bacteria belonging to Escherichia or Pseudomonas are preferable. Specific examples of the bacterium bound to an antibody in the removal method I of the present invention include Escherichia coli, preferably O26 strain, O91 strain, O103 strain, O111 strain, O115 strain, O121 strain, O128 strain, O145 strain, O157 strain and O159 strain. Specific examples of the bacterium belonging to Pseudomonas and bound to an antibody in the removal method of the present invention include Pseudomonas aeruginosa, preferably NCTC12924 strain. Examples of the gram-positive bacterium include, but are not limited to, bacteria belonging to Staphylococcus, Streptococcus, Enterococcus, Corynebacterium, Bacillus, Listeria, Peptococcus, Peptostreptococcus, Clostridium, Eubacterium, Propionibacterium, Lactobacillus and the like. Specific examples of the bacterium belonging to Streptococcus and bound to an antibody in the removal method I of the present invention include Streptococcus pneumoniae, preferably serotype 15B/C.

The cell bound to an antibody in the removal method I of the present invention is not particularly limited as long as it is a cell of a eucaryote, and is defined as a concept including animal cell, plant cell and fungi. Of these, an animal cell is preferable. Examples of the animal cell include, but are not limited to, hepatocyte, splenocyte, nerve cell, glial cell, pancreatic β cell, bone marrow cell, mesangial cell, Langerhans cell, epidermal cell, epithelial cell, goblet cell, endothelial cell, smooth muscle cell, fibroblast, fibroblast, muscle cell, adipocyte, immunocyte (e.g., macrophage, T cell, B cell, natural killer cell, mast cell, neutrophil, basophil, eosinophils, monocyte), megakaryocyte, synovial cell, chondrocyte, osteocyte, osteoblast, osteoclast, mammary cell, hepatocyte or interstitial cell, and progenitor cell, stem cell or cancer cell of these cells, and the like. Among these, mast cell is preferable.

The vesicle bound to an antibody in the removal method I of the present invention is not particularly limited as long as it has a lipid bilayer membrane as an outermost membrane. Examples thereof include exosome, extracellular vesicle, membrane vesicle, exosome-like vesicle and the like.

Examples of the virus bound to an antibody in the removal method I of the present invention include capsid type virus and envelope type virus.

Examples of the antibody to which the above-mentioned microorganism or the like are bound in the removal method I of the present invention include both polyclonal antibody and monoclonal antibody. The antibody may belong to any of the immunoglobulin classes of IgG, IgA, IgM, IgD and IgE, preferably IgG. As the antibody in the present invention, a commercially available antibody, an antibody stored in a research institute or the like that binds to the target microorganism may also be used. Alternatively, those of ordinary skill in the art can produce an antibody that binds to the object microorganism or the like as follows.

A polyclonal antibody can be produced by, for example, the following method. Using the above-mentioned microorganism itself or a part of the microorganism (e.g., O-antigen, F-antigen, H-antigen, K-antigen and the like for bacterium, membrane protein for animal cell, exosome, and the like) or the like as a sensitizing antigen, mammals such as rabbit, mouse, rat, goat, guinea pig, hamster and the like and birds such as chicken and the like are immunized (1 to several times of booster immunization every about 1-4 weeks after first immunization), partial blood sampling is performed about 3 to 10 days after each booster immunization, and the antibody titer of the collected serum is measured by utilizing a conventionally-known antigen-antibody reaction, and its increase is confirmed. About 3-10 days after the final immunization, whole blood is collected and the antiserum is purified. Polyclonal antibody can also be purified as a single immunoglobulin class by a conventionally-used separation technique such as salting out such as ammonium sulfate fraction and the like, centrifugation, dialysis, column chromatography and the like.

In addition, a monoclonal antibody can be obtained from hybridoma (fusion cell) generally produced by cell fusion. That is, as in the case of the above-mentioned polyclonal antibody, antibody producing cells are isolated from a mammal immunized with the above-mentioned sensitizing antigen, they are fused with myeloma cells to form hybridomas, the hybridomas are cloned, and a monoclonal antibody is produced by using the above-mentioned immunized antigen as a marker antigen and selecting a clone that produces an antibody exhibiting a specific affinity therefore. It is also possible to use antibody-producing cells produced by reacting the above-mentioned sensitizing antigen with previously-isolated splenocyte, lymphocyte and the like in a culture medium. In this case, antibody producing cells derived from human can also be prepared.

Hybridoma that secretes a monoclonal antibody can be prepared according to the method of Kohler and Milstein (Nature, Vol. 256, pp. 495-497, 1975) and a modified method thereof. That is, the monoclonal antibody of the present invention is prepared by culturing a hybridoma obtained by fusing an antibody producing cell contained in splenocyte, lymph node cell, peripheral lymphocyte, myeloma cell, tonsil cell and the like, preferably splenocyte, obtained from an animal immunized as mentioned above, and a myeloma cell (myeloma) of preferably mammal such as allogeneic mouse, rat, guinea pig, hamster, rabbit, human or the like, more preferably mouse, rat or human. Culturing can be carried out in vitro or in vivo (intraperitoneally) in mammals such as mouse, rat, guinea pig, hamster, rabbit and the like, preferably mouse or rat, more preferably mouse, and an antibody can be obtained from respective culture supernatant or ascites of a mammal.

Examples of the myeloma cell used for cell fusion include mouse-derived myeloma P3/X63-AG8, P3/NSI/1-Ag4-1, P3/X63-Ag8.U1, SP2/0-Ag14, F0 or BW5147, rat-derived myeloma 210RCY3-Ag1.2.3., human-derived myeloma U-266AR1, GML500-6TG-A1-2, UC729-6, CEM-AGR, D1R11 or CEM-T15 and the like.

A hybridoma clone producing monoclonal antibody can be screened for by, for example, culturing a hybridoma in a microtiter plate, and measuring reactivity of the culture supernatant in a well showing proliferation to a marker antigen by radioimmunoassay, enzyme immunoassay, fluorescence immunoassay and the like.

A monoclonal antibody can be isolated and purified by subjecting the antibody-containing culture supernatant or ascites produced by the aforementioned method to ion exchange chromatography or affinity column chromatography such as anti-immunoglobulin column, protein A column and the like.

The monoclonal antibody may be obtained by any method without limitation to the aforementioned production method. Generally, monoclonal antibodies have sugar chains having different structures depending on the type of mammal to which immunization is applied. The monoclonal antibody in the present invention is not limited by the structural difference of the sugar chain, and encompasses monoclonal antibody derived from any mammal. Furthermore, for example, a recombinant human-type monoclonal antibody obtained from a transgenic animal into which a human immunoglobulin gene is integrated, a chimeric monoclonal antibody in which the constant region (Fc) of a monoclonal antibody derived from a certain mammal is recombined with the Fc region of a human-derived monoclonal antibody, and a chimeric monoclonal antibody in which the whole region other than the complementarity determining region (CDR) capable of directly binding to the antigen in a complementary manner is recombined with the corresponding region of a human-derived monoclonal antibody are also encompassed in the above-mentioned monoclonal antibody.

In the removal method I of the present invention, the antibody to which microorganism or the like are bound includes naturally-occurring antibodies such as the aforementioned polyclonal antibody, monoclonal antibody (mAb) and the like, a chimeric antibody that can be produced using a gene recombination technique, a humanized antibody, a single-stranded antibody, and fragments of these antibodies. A fragment of an antibody means a partial region of the aforementioned antibody having a specific binding activity and specifically encompasses Fab, Fab′, F(ab′)2, scAb, scFv, scFv-Fc and the like.

In addition, those of ordinary skill in the art can prepare a fusion antibody of the above-mentioned antibody or fragment and other peptide or protein, or a modified antibody with a modulating agent bonded thereto. Other peptide and protein used for fusion are not particularly limited as long as they do not lower the binding activity of the antibody. Examples thereof include human serum albumin, various tag peptides, artificial helix motif peptide, maltose binding protein, glutathione S transferase, various toxins, other peptide and protein capable of promoting multimerization and the like. A modulating agent used for modification is not particularly limited as long as they do not lower the binding activity of the antibody. Examples thereof include polyethylene glycol, sugar chain, phospholipid, liposome, low-molecular-weight compound and the like.

The antibody used in the removal method I of the present invention is characteristically immobilized on a carrier. The carrier is not particularly limited as long as it can be used for immunological detection methods. Examples thereof include synthetic resin such as polystyrene, polyacrylamide, silicon and the like, glass, metal thin film, nitrocellulose membrane and the like. For immobilization of an antibody on a carrier, physical adsorption may be used, or a method using a chemical bond generally used for insolubilizing and immobilizing protein, enzyme and the like may be applied.

In the removal method I of the present invention, the primary gel of the present invention can remove microorganism or the like as an antigen from an antibody when the dispersoid is crosslinked or associated to lose flowability and it has a size and a shape free from clogging of the flow pathway and reactor. However, a gel having a certain range of hardness can be more efficiently remove microorganism or the like from the antibody. The hardness in the present invention can be defined by the breaking strength. The breaking strength here is defined as a force (g/cm²) required to break a gel prepared in a disc shape with a diameter of 100 mm and a thickness of 10 mm by compressing the gel by lowering a plunger with a cross-section area of 2.0 cm² at a rate of 0.8 mm per second by using a Texo Graph. Generally, the gel used in the removal method I of the present invention may have any breaking strength value, and it is preferably 4-1, 100 g/cm², more preferably, 8-1, 100 g/cm². When the breaking strength of the gel is within the range of 4-1, 100 g/cm², it can be efficiently removed from an antibody such as microorganism or the like, and more efficiently removed when it is within the range of 8-1, 100 g/cm².

In the removal method I of the present invention, the flow rate, flow time, temperature during flowing of the primary gel of the present invention can be appropriately determined by those of ordinary skill in the art.

For example, the flow rate of the primary gel of the present invention is generally 100 μm/s-100 mm/s, preferably 500 μm/s-25 mm/s.

For example, the flow time of the primary gel of the present invention is generally 30 sec-1200 sec, preferably 60 sec-480 sec.

For example, the temperature during flowing of the primary gel of the present invention is generally 4° C.-37° C., preferably 15° C.-30° C.

The primary gel of the present invention may be a gel of polysaccharide, protein or synthetic polymer. Examples of the polysaccharides used for the primary gel of the present invention include agarose, agar, carrageenan, pectin, sodium alginate, glucomannan, gellan gum, xanthan gum, locust bean gum, tamarind seed gum, curdlan and the like, with preference given to agarose, agar and carrageenan. Examples of the protein used for the primary gel of the present invention include gelatin, soybean casein, fibrin, albumen protein, whey protein and the like, with preference given to gelatin. Examples of the synthetic polymer used for the primary gel of the present invention include polyacrylamide, sodium polyacrylate, polyvinyl chloride, polyvinyl alcohol and the like.

The gel can be produced by a known method. For example, agarose gel can be prepared by producing an agarose solution by adding an agarose powder to distilled water and boiling same, diluting the agarose solution with heated distilled water to a desired concentration, and cooling same to 4° C. Gelatin gel can be prepared by producing a gelatin solution by adding a gelatin powder to heated distilled water, diluting the gelatin solution with heated distilled water to a desired concentration, and cooling same to 4° C. Agar gel can be prepared by producing an agar solution by adding an agar powder to heated distilled water and boiling same, diluting the agar solution with heated distilled water to a desired concentration, and cooling same to 4° C. Carrageenan gel can be prepared by producing a carrageenan solution by adding a carrageenan powder to heated distilled water and boiling same, diluting the carrageenan solution with heated aqueous potassium chloride solution to a desired concentration, and cooling same to 4° C.

In the removal method I of the present invention, the carrier surface may be washed with a wash buffer before, after, or both before and after flowing the primary gel of the invention on the carrier surface. The wash buffer is not particularly limited as long as it is a solvent that dissolves the test sample in the below-mentioned removal method II of the present invention and is a physiological salt solution suitable for an antigen-antibody reaction. Examples thereof include, but are not limited to, PBS containing 0.2% BSA and 0.02% Tween20, PBS containing 0.2% BSA, 0.02% Tween20 and 5 mmol/L EDTA, D-PBS(−) containing 0.1% Casein and the like. The washing rate, washing time and temperature during washing of the wash buffer of the present invention can be appropriately determined by those of ordinary skill in the art.

As mentioned above, by flowing a gel on the surface of a carrier immobilized with an antibody to which a microorganism or the like as an antigen is bound, the microorganism or the like can be removed from the antibody. However, the binding between the microorganism or the like and the antibody depends on the combination of the microorganism or the like and the antibody. Therefore, even when the removal method I of the present invention is used, the microorganism or the like may be removed from the antibody only partially. The present inventors successfully removed even the microorganism or the like that cannot be removed even by the removal method I of the present invention, by utilizing the salting out effect by a salt aqueous solution in addition to the gel.

Therefore, the present invention also provides a method for removing microorganism or the like as an antigen from an antibody immobilized on a carrier and to which the microorganism or the like has been bound, the method comprising flowing a mixture containing the primary gel of the present invention and a salt aqueous solution on a surface of the carrier, washing same and thereafter flowing again a gel the same as or different from the primary gel of the present invention (hereinafter sometimes to be also indicated as secondary gel of the present invention) (hereinafter sometimes to be indicated as the removal method II of the present invention).

As a microorganism, a cell, a vesicle secreted by the microorganism or the cell, or a virus bound to the antibody in the removal method II of the present invention may be similar to the microorganism or the like described in the removal method I of the present invention. In the case of a microorganism, a bacterium belonging to Escherichia, Pseudomonas or Streptococcus is preferable. Of these, as the bacterium belonging to Escherichia, Escherichia coli can be mentioned, and preferred are O26 strain, O91 strain, O103 strain, O111 strain, O115 strain, O121 strain, O128 strain, O145 strain, O157 strain and O159 strain. Examples of the bacterium belonging to Pseudomonas include Pseudomonas aeruginosa, preferably NCTC12924 strain. As the specific bacterium belonging to Streptococcus, Streptococcus pneumoniae is more preferable, and serotype 15B/C or 35B is more preferable. In the case of a cell, an animal cell is preferable, and mast cell is more preferable. In the case of a vesicle, exosome is preferable.

In the removal method II of the present invention, the antibody to which the above-mentioned microorganism or the like are bound and the carrier immobilized with the antibody may be the same as the antibody and carrier described in the removal method I of the present invention.

The primary gel of the present invention and the secondary gel of the present invention used in the removal method II of the present invention may be similar to the gel described in the removal method I of the present invention. The primary gel of the present invention and the secondary gel of the present invention may be the same gel or a different gel. When the primary gel of the present invention is an agarose gel, the secondary gel of the present invention is preferably a gelatin gel. In the removal method II of the present invention, hardness of the gel, flow rate, flow time, and temperature during flowing of the gel flown on the carrier surface may be the same as the conditions in the removal method I of the present invention.

A salt aqueous solution to be mixed with the primary gel of the present invention used in the removal method II of the present invention is not particularly limited as long as it is a salt aqueous solution capable of eluting the microorganism or the like by an ion salting out effect. Examples of the salt include a salt of a combination of CO₃²⁻, SO₄²⁻, H₂PO₄⁻ and NH₄⁺, K⁺, Na⁺, standing on top of the so-called Hofmeister series, and the like, and preferred is ammonium sulfate. The concentration of the salt aqueous solution is not limited as long as it can afford a salting out effect, and a saturated salt aqueous solution is preferable. The primary gel of the present invention and the salt solution may be mixed at any quantitative ratio, and equal amounts are preferably mixed since handling is easy.

In the removal method II of the present invention, the carrier surface may be washed with a mixture of the primary gel of the present invention and a salt aqueous solution before, after, or both before and after flowing the mixture on the carrier surface. The washing rate, washing time and temperature during washing of the salt aqueous solution of the present invention can be appropriately determined by those of ordinary skill in the art.

In the removal method II of the present invention, the carrier surface may be washed with a wash buffer before, after, or both before and after flowing the secondary gel of the invention on the carrier surface. The composition of the wash buffer, washing rate, washing time and temperature during washing of the wash buffer may be similar to the conditions in the removal method I of the present invention.

Since microorganism or the like are somewhat large relative to the antibody, they are considered to be bound at multiple sites. In this case, the microorganism or the like cannot be removed from the antibody by only a chemical means such as changing pH and the like. However, it is considered that an action to physically detach the microorganism or the like from the antibody is caused by flowing the gel on the surface of the carrier on which the antibody is immobilized. In addition, the removal method I of the present invention (or the removal method II of the present invention) can be applied not only to an antibody bound to the microorganism or the like but also to an antibody bound to a protein.

As mentioned above, a carrier immobilized with an antibody from which the microorganism or the like have been removed in the removal method I of the present invention (or the removal method II of the present invention) can be repeatedly used for immunological detection of the microorganism or the like without disposing. Therefore, the present invention provides an immunological detection method of the microorganism or the like, comprising flowing the primary gel of the present invention on the surface of a carrier immobilized with an antibody to which a microorganism or the like as an antigen is bound, thus removing the microorganism or the like from the antibody, contacting a new test sample with the carrier, and detecting the microorganism or the like by an immunological method. Furthermore, the present invention also provides an immunological detection method of the microorganism or the like, comprising flowing a mixture of the primary gel of the present invention and a salt aqueous solution on the surface of a carrier immobilized with an antibody to which a microorganism or the like as an antigen is bound, washing, and flowing again the secondary gel of the present invention which is the same as or different from the primary gel of the present invention, thus removing the microorganism or the like from the antibody, contacting a new test sample with the carrier, and detecting the microorganism or the like by an immunological method (hereinafter sometimes to be indicated as the immunological detection method of the present invention).

A test sample used in the immunological detection method of the present invention is not particularly limited as long as it contains a microorganism or the like to be the target of trapping by the antibody immobilized on a carrier in the removal method I of the present invention (or the removal method II of the present invention). Examples thereof include foods, blood sample, saliva, urine, feces, body fluid, cell culture medium, cultured cells, culture microorganism, environment water, soil and the like.

The immunological method used in the immunological detection method of the present invention is not particularly limited as long as it is an immunological detection method for detecting a microorganism or the like—antibody complex composed of microorganism or the like in a test sample and the corresponding antibody by a chemical or physical means, and any measurement method may also be used. In addition, the amount of the microorganism or the like can also be calculated as necessary from a standard curve drawn using a standard solution containing a known amount of the microorganism or the like. As the immunological method used in the immunological detection method of the present invention, any method may be used as long as an antigen-antibody reaction is carried out on the surface of a solid phase, irrespective of a batch system or a flow system, such as ELISA, immunity sensor and the like.

As a labeling agent used for a measurement method using a labeling substance, radioisotope, enzyme, fluorescent substance, luminescence substance and the like are used. As the radioisotope, [¹²⁵I], [¹³¹I], [³H], [¹⁴C] and the like are used. As the above-mentioned enzyme, one which is stable and having high specific activity is preferable and, for example, β-galactosidase, β-glucosidase, alkaline phosphatase, peroxidase, malic acid dehydrogenase and the like are used. As the fluorescent substance, fluorescamine, fluorescein isothiocyanate and the like are used. As the luminescence substance, luminol, luminol derivative, luciferin, lucigenin and the like are used. In addition, a biotin-avidin system can also be used for binding an antibody and a label.

In a sandwich method, a sample possibly containing a microorganism or the like is reacted with an antibody immobilized on a carrier (primary reaction), a labeled secondary antibody to the microorganism or the like is reacted (secondary reaction), and the amount (activity) of the label on the carrier is measured, whereby the microorganism or the like in the sample can be detected and quantified. The primary reaction and the secondary reaction may be performed in a reverse order or performed simultaneously or at different times.

Alternatively, using an immunity sensor by a surface plasmon resonance (SPR) method, an antibody is immobilized on the surface of a commercially available sensor chip according to a conventional method, it is contacted with a sample possibly containing the microorganism or the like, a light with a particular wavelength is irradiated to the sensor chip from a particular angle, and the presence or absence of binding of a microorganism or the like to the immobilized antibody can be determined with the change in the resonance angle as an index.

As mentioned above, a carrier immobilized with the antibody can be repeatedly reutilized by flowing a gel on the surface of a carrier immobilized with an antibody to which a microorganism or the like as an antigen is bound, thus removing the microorganism or the like from the antibody, rebinding other microorganism or the like contained in the test sample to the antibody, and detecting the microorganism or the like by the above-mentioned immunological method.

The present invention also provides an apparatus for removing a microorganism, a cell, a vesicle secreted by the microorganism or the cell, or a virus, comprising a mechanism for flowing a gel on a surface of a carrier immobilized with an antibody to which the microorganism or the like as an antigen are bound (hereinafter sometimes to be indicated as the apparatus of the present invention).

In the apparatus of the present invention, the microorganism or the like bound to the antibody, the antibody to which the microorganism or the like are bound, and the carrier immobilized with the antibody may be similar to the microorganism or the like, antibody and carrier described in the removal method I of the present invention (or the removal method II of the present invention) and the immunological detection method of the present invention.

The gel used in the apparatus of the present invention may be similar to gel described in the removal method I of the present invention (or the removal method II of the present invention) and the immunological detection method of the present invention. The hardness of the gel, flow rate, flow time, and temperature during flowing of the gel flown on the carrier surface may be the same as the conditions described in the removal method I of the present invention (or the removal method II of the present invention), and the immunological detection method of the present invention. In the apparatus of the present invention, the primary gel and the secondary gel of the present invention may be supplied in order as in the removal methods I and II of the present invention.

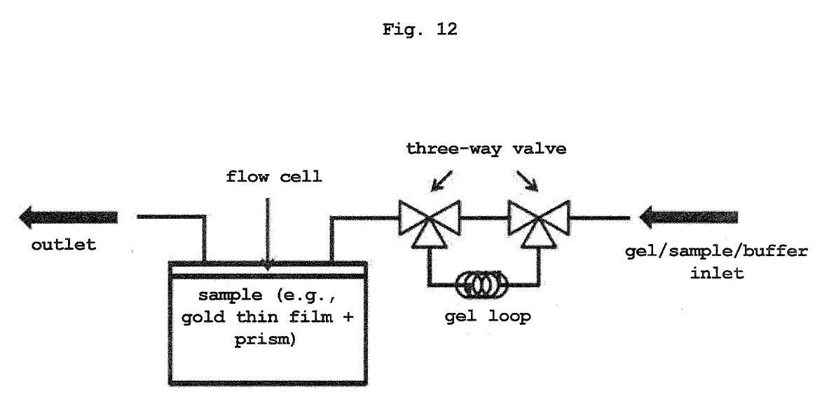

In the apparatus of the present invention, the mechanism for flowing a gel on a carrier surface (hereinafter sometimes to be indicated as the mechanism of the present invention) is not particularly limited as long as it can flow a gel on a carrier surface. For example, as shown in FIG. 12, the mechanism of the present invention contains an inlet for gel/sample/buffer, a carrier composed of a metal film and a prism on which a flow cell is provided, and an outlet. In the mechanism of the present invention, a test sample, wash buffer and gel are supplied from the inlet, they are pushed out to the carrier provided with a flow cell and are discharged from the outlet. More specifically, a test sample supplied from the inlet contacts the carrier surface, a microorganism or the like contained in the test sample is trapped by an antibody immobilized on a carrier. Then, a wash buffer is supplied from the inlet, and discharged from the outlet together with the test sample. The gel is supplied from the inlet and flows on the carrier surface to remove the microorganism or the like trapped by the antibody immobilized on the carrier. Finally, the wash buffer is supplied from the inlet and discharged from the outlet together with the gel. In this case, the supply pressure may be adjusted such that the gel will be supplied at a desired flow rate, flow time and temperature on the carrier surface. Therefore, to adjust the supply pressure of the gel, the mechanism of the present invention may be further provided with a three-way valve and a gel loop, as shown in FIG. 12. Of the three valves of the three-way valve, only the valve connected to the flow path on the gel loop side is opened to retain the gel in the gel loop, and the supply pressure can be adjusted so that the gel on the carrier surface can be supplied at a desired flow rate, flow time and temperature.

The apparatus of the present invention may include a mechanism for continuously observing whether or not a microorganism or the like is trapped in an antibody immobilized on a carrier. Examples of such mechanism include a light source for SPR method, a reflection light detector and a reflection light analysis apparatus. By this mechanism, whether or not a microorganism or the like is bound to an antibody immobilized on a carrier can be detected in real time.

### EXAMPLES

The present invention is explained in more detail in the following by referring to Examples, which are not to be construed as limitative.

### Construction of Immunosensor by Surface Plasmon Resonance (SPR)

An immunosensor by SPR was constructed using a microarray type SPRi apparatus (Horiba, Ltd.: OpenPlex), biochip dedicated to the apparatus (Horiba, Ltd.: CS-HD), and 10 kinds of rabbit antiserum to O-antigen of Escherichia coli (DENKA SEIKEN Co., LTD: enteropathogenic Escherichia coli immune serum “SEIKEN” O111, O157, O26, O91, O103, O115, O121, O128, O145, O159), 2 kinds of rabbit pool antiserum to capsular antigen of Streptococcus pneumoniae (Statens Serum Institut: Pneumococcus Pool Antisera Type G, Type S), monoclonal antibody CD9 to exosome CD9 (R&D system, Inc., MAB1880), Human/Mouse CD117/c-Kit antibody to mouse mast cell CD117 (R&D systems Inc., AF1356), and immune serum “SEIKEN” I group for Pseudomonas aeruginosa group (DENKA SEIKEN Co., LTD, 213662). As for antiserum, antibody was purified in advance from each antiserum by using protein G (GE Healthcare: Protein G Sepharose 4 Fast Flow) according to the manual. The prepared antibody was immobilized on a biochip according to the manual of CS-HD to prepare a sensor chip. The chip was mounted on the SPRi apparatus and used as an immunosensor for the detection of microorganism or the like.

### Preparation of Gel

### 1. Gelatin Gel

Gelatin (Nacalai Tesque: purified powder) (50 g) was dissolved in distilled water (500 mL) heated to 85° C. to prepare a 10% gelatin solution. This was diluted with distilled water heated to 85° C. to 10, 8, 6, 4, 3, 2, 1.5, 1.3, 1.1, 1.0, 0.7, 0.5% and stood at 4° C. for 4 days to give gelatin gel with each concentration.

### 2. Agarose Gel

Agarose (Nacalai Tesque: low electro-osmosis, high gel strength) (15 g) was added to distilled water (500 mL) and boiled to prepare a 3% agarose solution. This was diluted with distilled water heated to 85° C. to 3, 2, 1, 0.8, 0.6, 0.4, 0.2, 0.1, 0.05, 0.025, 0.0125% and stood at 4° C. for 1 hr to give agarose gel with each concentration.

### 3. Agar Gel

Purified agar powder (Nacalai Tesque: for microorganism culture) (1 g) was added to distilled water (500 mL), dissolved by boiling and stood at 4° C. for 1 hr to give 0.2% agar gel.

### 4. Carrageenan Gel

Carrageenan (Nacalai Tesque) (1 g) was added to distilled water (500 mL), dissolved by boiling, 3.5 mol/L potassium chloride (12.5 mL) was added and the mixture was stood at 4° C. for 1 hr to give 0.2% carrageenan gel.

### Example 1 Detection of O-Antigen of Escherichia coli and Regeneration of Sensor Chip (1)

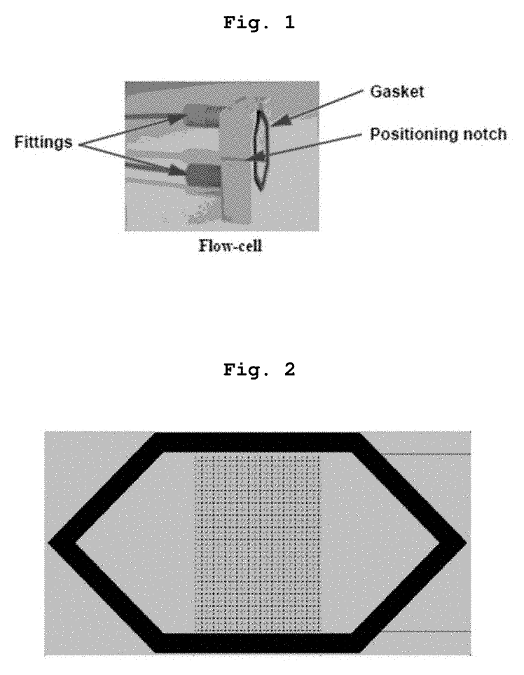

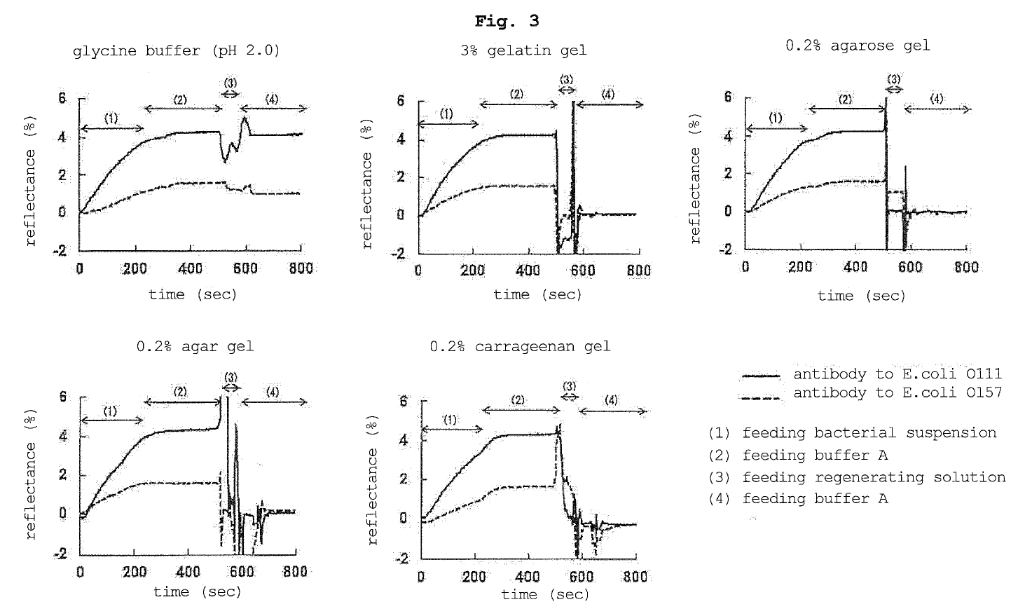

Two strains showing the serotypes of O111 and O157 were used as Escherichia coli. The constructed immunosensor can measure every 3 seconds the change amount of reflected light caused by the SPR phenomenon induced by the binding of molecules to the sensor chip surface as reflectance (%). Buffer, sample or regenerating solution was contacted with the sensor chip surface via Flow-cell (FIG. 1). Flow-cell is fixed in contact with the sensor chip in a position (FIG. 2) where the entire Gasket is completely covered by the sensor chip. Among the flat planes of Flow-cell, the flat plane surrounded by the frame of Gasket is recessed by 80 μm than the flat plane of the periphery of the Gasket frame. As a result, in the sensor chip in contact with Flow-cell, a spatial gap of 80 μm in width is generated between the flat plane surrounded by the Gasket frame of the Flow-cell and the sensor chip surface. Therefore, a buffer or the like fed from one polyvinyl chloride tube (inner diameter 380 μm) connected to the Flow-cell via Fitting contacts the surface of the sensor chip by filling the spatial gap of 80 μm in width, and excreted from the other polyvinyl chloride tube. Prior to the measurement, the surface of the sensor chip with an antibody immobilized thereon was conditioned by feeding buffer A (PBS containing 0.2% BSA and 0.02% Tween 20) at a flow rate of 50 μL/min. The measurement was started with the reflectance at this point as 0%. First, two bacterial strains were suspended in Buffer A and fed to the surface of the sensor chip for 240 sec, and then buffer A was fed for 260 sec. The reflectance at this point was 4.3% for O111 and 1.6% for O157, and the bacterial cells were bound to the immobilized antibody on the chip (FIGS. 3-(1) and -(2)).

Next, regeneration of the sensor chip, that is, removal of the bacterial cells bound to the immobilized antibody was tried. For regeneration, it is required that the bacterial cells can be completely or partially removed, and that immobilized antibody is not influenced. A 100 mmol/L glycine buffer (pH 2.0) as a regeneration solution of a conventional method, and 3% gelatin gel, 0.2% agarose gel, 0.2% agar gel, 0.2% carrageenan gel as regeneration solutions for the new trial were each fed for 60 sec (FIG. 3-(3)). To prevent dissolution at room temperature and maintain gel strength, 3% gelatin gel was ice-cooled. Next, buffer A was fed for 240 sec and the regenerating solution remaining on the chip surface was removed. As is clear from FIG. 3-(4), after feeding 100 mmol/L glycine buffer (pH 2.0), the reflectance of O111 was 4.1% and that of O157 was 1.0% and could not return to 0% at the start of the measurement. Thus, bacterial cells were hardly removed. On the other hand, after feeding 3% gelatin gel, 0.2% agarose gel, 0.2% agar gel or 0.2% carrageenan gel, the reflectance converged to 0% and O111 and O157 could be completely removed. In addition, the experiment was conducted again using the chips after regeneration, and the original reflectance was reproduced. From these results, it was clarified that 3% gelatin gel, 0.2% agarose gel, 0.2% agar gel and 0.2% carrageenan gel can preferably regenerate the sensor chip.

### Example 2 Detection of Capsular Antigen of Streptococcus pneumoniae and Regeneration of Sensor Chip

Two strains showing the serotypes of 15B/C, 35B were used as Streptococcus pneumoniae. 15B/C shows a coagulation reaction with Type S antiserum, and the 35B shows a coagulation reaction with Type G antiserum.

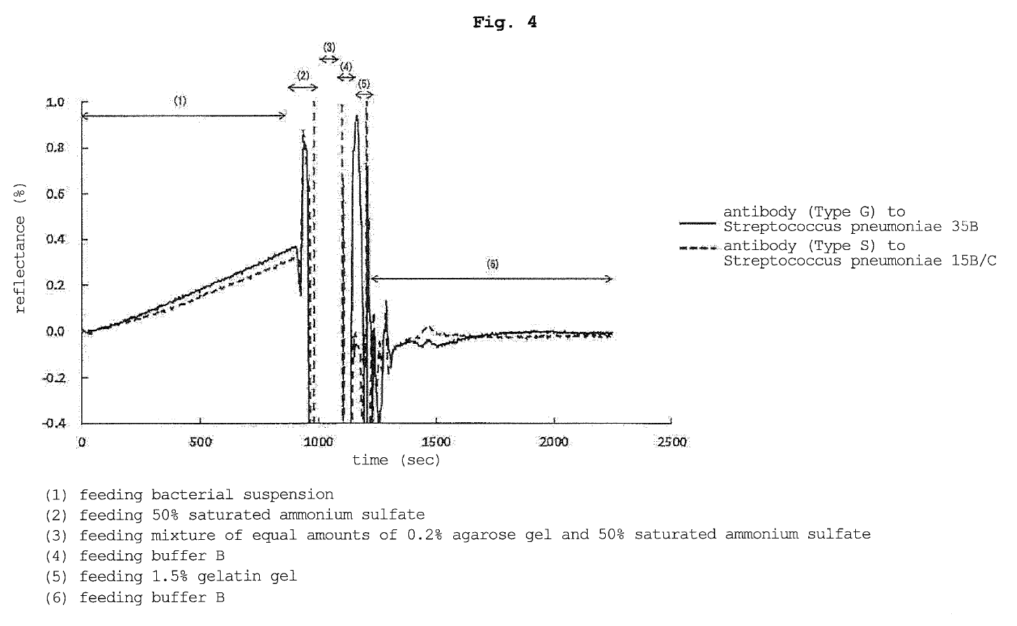

Prior to the measurement, the surface of the sensor chip with the antibody immobilized thereon was conditioned by feeding buffer B (PBS containing 0.2% BSA, 0.02% Tween 20 and 5 mmol/L EDTA) at a flow rate of 50 μL/min. The measurement was started with the reflectance at this point as 0%. First, two bacterial strains were suspended in Buffer B and fed to the surface of the sensor chip. The binding rate of Streptococcus pneumoniae and the antibody is late and the binding is inhibited by the liquid flow. Therefore, the liquid feeding was stopped, and the bacterial suspension was kept on the chip surface for 900 sec to bind the bacterial cells and the antibody. The reflectance at this point was 0.36% for Type G and 0.32% for Type S, and the bacterial cells were bound to the antibody immobilized on the chip (FIG. 4-(1)).

Next, regeneration of sensor chip was tried. Regeneration method using 3% gelatin gel was tried in the same manner as the above-mentioned operation performed after detection of E. coli O-antigen. As a result, the reflectance of Type S converged to 0%; however, the reflectance of Type G was only 0.14-0.31%, and the regeneration of the sensor chip was partial. Therefrom it was found that Streptococcus pneumoniae shows stronger binding to the antibody than Escherichia coli depending on the strain. Therefore, attempts were made to remove the bacterial cells not only by the gel regeneration effect but also by combining a salting-out effect by ammonium sulfate.

That is, after detection, 50% saturated ammonium sulfate was fed for 120 sec at a flow rate of 9000 μL/min, and then a mixture of equal amounts of 0.2% agarose gel and 50% saturated ammonium sulfate was fed for 60 sec at a flow rate of 50 μL/min (FIGS. 4-(2) and -(3)). As a result, the reflectance was 0.07-0.08% for Type G and 0% for Type S. In Type S, the bacterial cells could be removed; however, a part of the bacterium of type G and the agarose gel remained on the chip surface and could not be regenerated completely. However, these could be removed by feeding buffer B for 60 sec at a flow rate of 9000 μL/min and feeding ice-cooled 1.5% gelatin gel for 60 sec (FIGS. 4-(4) and -(5)). Finally, buffer B was fed for 1000 sec at a flow rate of 50 μL/min (FIG. 4-(6)). Complete removal of the microbial cells was clear from the reflectance of both Type G and Type S that was converged to 0%. In addition, the experiment was conducted again using the chips after regeneration, and the original reflectance was reproduced. Therefore, it was clarified that the immobilized antibody was free from influence. Also, to ascertain the regeneration effect by salting out, the experiment was conducted only with 50% saturated ammonium sulfate. As a result, the reflectance was not different from that at the time of detection, and regeneration was not possible. Regeneration of the chip by using a gel was possible even in the case of Streptococcus pneumoniae, and a combination of agarose and gelatin gels was particularly effective.

### Example 3 Relationship Between Gel Strength and Regeneration Effect

The breaking strength was measured using gelatin gel and agarose gel prepared in a disc shape with a diameter of 100 mm and a thickness of 10 mm by a preparation method as in the above. For the measurement, Texo Graph (Japan Food development laboratory: production NO. 9904-053) was used and a force (g/cm²) required to break the gel by compression was measured. For compression, a plunger with cross-sectional area of 2.0 cm² was lowered at 0.8 mm/sec. The regeneration effect of the gel was the value calculated by the following formula when detecting and regenerating Escherichia coli O111 and O157, and a regeneration effect in the range of 80-120% was defined to indicate successful regeneration.

(reflectance at detection of O-antigen−reflectance after chip regeneration by gel)/reflectance at detection of O-antigen×100=regeneration effect (%)

The measured gel strength and regeneration effect are shown in Table 1. The regeneration effect of 1.0-6% gelatin gel and 0.1-1% agarose gel was 88-116% for both O111, O157. The gel strength was 8-1064 g/cm² for gelatin gel and 4-1037 g/cm² for agarose gel, and it was found that these strengths can be used for regenerating chips.

Also, the gel strength could not be measured at 0.5-0.7% gelatin gel and 0.0125-0.05% agarose gel since they were below the measurement lower limit of Texo Graph. Even 0.5% gelatin gel and 0.0125% agarose gel with the lowest concentration showed a high regeneration effect for O157 and were found to be usable for chip regeneration.

The 2-3% agarose gel and 8-10% gelatin gel have high gel strength and cause clogging in the flow path, which prevents feeding. The constructed immunosensor could not be used but is expected to have a regenerating effect.

TABLE 1

| | | | regenerating effect | |
|---|---|---|---|---|
| | concentration | breaking strength | (%) | |
| gel | (%) | (g/cm²) | O111 | O157 |
| gelatin gel | 0.5 | ND | 15 | 91 |
| (ice-cooled) | 0.7 | ND | 50 | 112 |
| | 1.0 | 8 | 91 | 97 |
| | 1.1 | 22 | 110 | 105 |
| | 1.3 | 44 | NT | NT |
| | 1.5 | 73 | NT | NT |
| | 2 | 165 | 100 | 99 |
| | 3 | 319 | 100 | 99 |
| | 4 | 497 | 105 | 100 |
| | 6 | 1064 | 116 | 112 |
| | 8 | 1423 | ND | ND |
| | 10 | 2059 | ND | ND |
| agarose gel | 0.0125 | ND | 11 | 72 |
| (25° C.) | 0.025 | ND | 31 | 89 |
| | 0.05 | ND | 92 | 100 |
| | 0.1 | 4 | 112 | 100 |
| | 0.2 | 21 | 100 | 103 |
| | 0.4 | 147 | NT | NT |
| | 0.6 | 382 | NT | NT |
| | 0.8 | 670 | NT | NT |
| | 1 | 1038 | 92 | 88 |
| | 2 | 2448 | ND | ND |
| | 3 | 2403 | ND | ND |

NT: Not test,
ND: Not detection (due to clogging of gel in measuring apparatus)

### Example 4 Detection of O-Antigen of Escherichia coli and Regeneration of Sensor Chip (2)

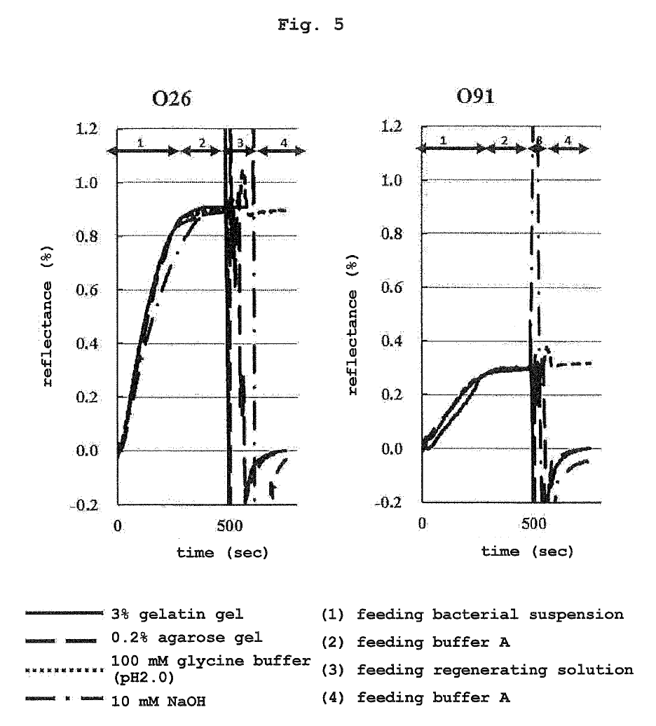

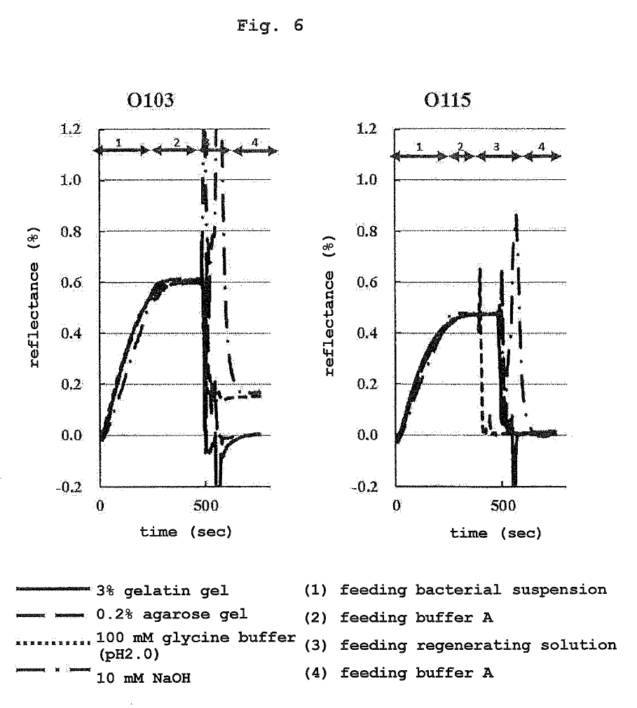

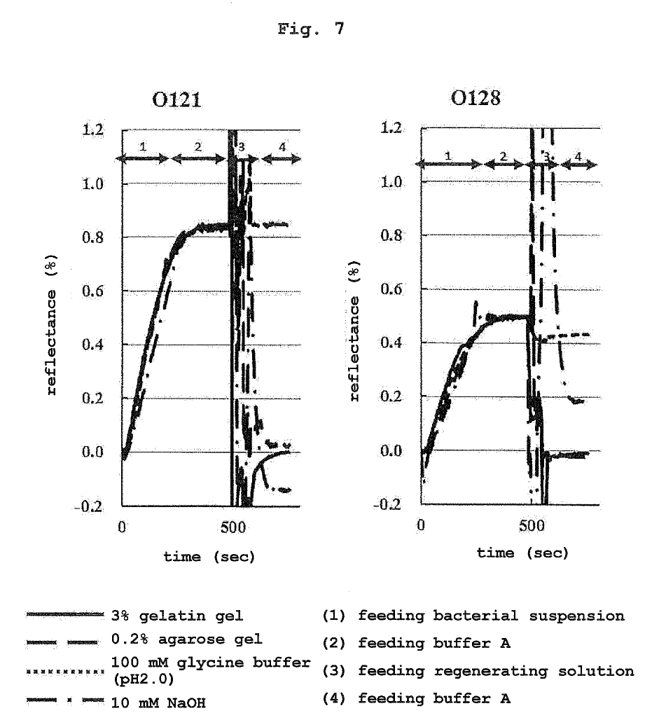

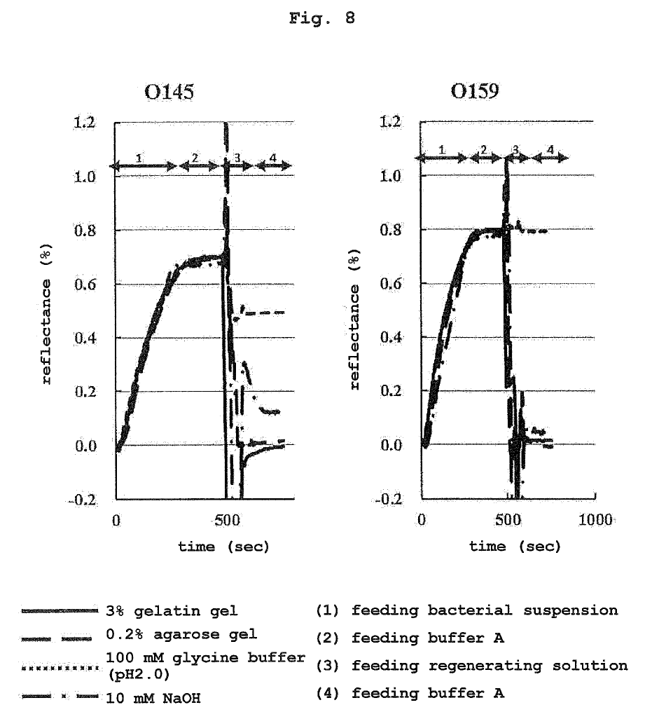

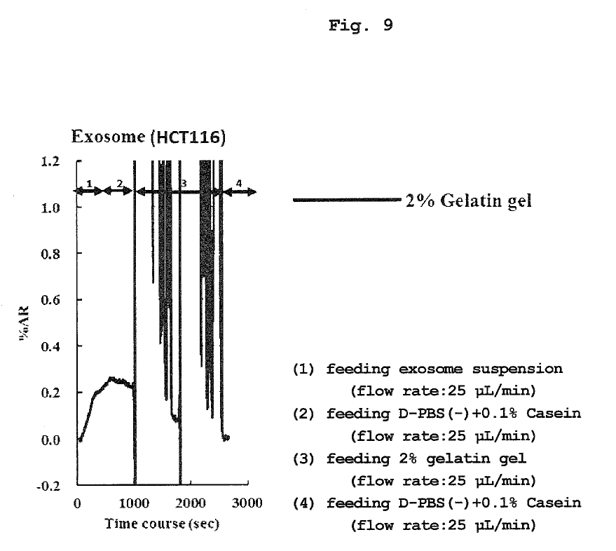

Eight strains showing the serotypes of O26, O91, O103, O115, O121, O128, O145, O159 were used as Escherichia coli. In the same manner as in Example 1, prior to the measurement, the surface of the sensor chip with the antibody immobilized thereon was conditioned by feeding buffer A (PBS containing 0.2% BSA and 0.02% Tween 20) at a flow rate of 50 μL/min. The measurement was started with the reflectance at this point as 0%. First, eight bacterial strains were successively suspended in Buffer A and respectively fed to the surface of the sensor chip for 240 sec, and then buffer A was fed for 260 sec. The reflectance at this point was 0.9% for O26, 0.3% for O91, 0.6% for O103, 0.5% for O115, 0.8% for O121, 0.5% for O128, 0.7% for O145 and 0.8% for O159, and the bacterial cells were bound to the immobilized antibody on the chip (FIGS. 5-8).

Next, regeneration of the sensor chip, that is, removal of the bacterial cells bound to the immobilized antibody was tried. For regeneration, it is required that the bacterial cells can be completely or partially removed, and that immobilized antibody is not influenced. A 100 mmol/L glycine buffer (pH 2.0), 10 mM NaOH as regeneration solutions of a conventional method, and 3% gelatin gel and 0.2% agarose gel as regeneration solutions for the new trial were each fed for 60 sec. To prevent dissolution at room temperature and maintain gel strength, 3% gelatin gel was ice-cooled. Next, buffer A was fed for 240 sec and the regenerating solution remaining on the chip surface was removed. As is clear from FIGS. 5-8, after feeding 100 mmol/L glycine buffer (pH 2.0), the reflectance of all strains other than O115 could not return to 0% at the start of the measurement. Thus, bacterial cells were hardly removed. Even after feeding 10 mM NaOH, in all strains other than O115, the reflectance could not return to 0% at the start of the measurement. Conversely, when 10 mM NaOH was used, the reflectance decreased to less than 0% in O26, O91, O121 due to partial denaturation of the immobilized antibody. On the other hand, after feeding 3% gelatin gel or 0.2% agarose gel, the reflectance converged to 0% in 8 strains and the bacterial cells could be completely removed. From these results, it was clarified that 3% gelatin gel and 0.2% agarose gel can preferably regenerate the sensor chip.

### Example 5 Detection of Human Bowel Cancer-Derived Cell (HCT116)-Released Exosome and Regeneration of Sensor Chip

HCT116-released exosome was prepared by an ultracentrifugation method. In the same manner as in Example 1, prior to the measurement, the surface of the sensor chip with a CD9 monoclonal antibody immobilized thereon was conditioned by feeding buffer C (D-PBS(−) containing 0.1% Casein) at a flow rate of 25 μL/min. The measurement was started with the reflectance at this point as 0%. The HCT116-derived exosome was diluted 10-fold with buffer A and fed to the surface of the sensor chip for 480 sec, and then buffer A was fed for 480 sec. The reflectance at this point was 0.25%, and the exosome was bound to the immobilized antibody on the chip (FIG. 9).

Next, regeneration of the sensor chip, that is, removal of the exosome bound to the immobilized antibody was tried. For removal of the exosome, 2% gelatin gel was used. Similar to other Examples, 2% gelatin gel was ice-cooled to prevent dissolution at room temperature and maintain gel strength. The 2% gelatin gel was fed twice at 480 sec/one time. Thereafter, buffer A was fed for 360 sec, and the regenerating solution remaining on the chip surface was removed. As a result, after feeding 2% gelatin gel twice, the reflectance converged to 0% in the exosome as well, and the exosome could be completely removed.

### Example 6 Detection of Bone Narrow Derived Mast Cell and Regeneration of Sensor Chip

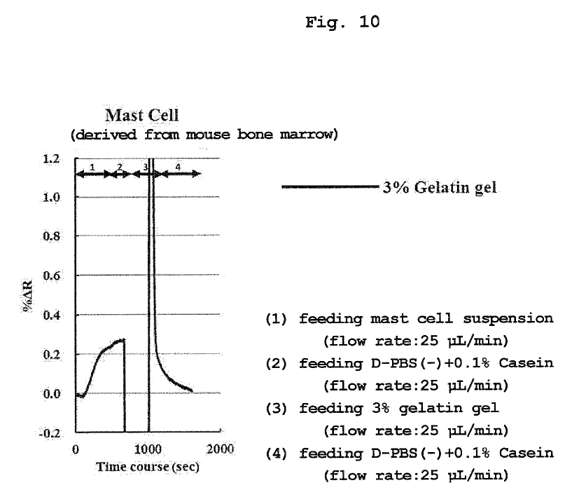

Mast cells were cultured in RPMI1640 medium containing 10% fetal bovine serum and interleukin-3. In the same manner as in Example 1, prior to the measurement, the surface of the sensor chip with a CD117/c-Kit antibody immobilized thereon was conditioned by feeding buffer C (D-PBS(−) containing 0.1% Casein) at a flow rate of 25 μL/min. The measurement was started with the reflectance at this point as 0%. The mast cells suspended in buffer A were fed to the surface of the sensor chip for 480 sec, and then buffer A was fed for 200 sec. The reflectance at this point was 0.27%, and the mast cells were specifically bound to the immobilized antibody on the chip (FIG. 10).

Next, regeneration of the sensor chip, that is, removal of the mast cells bound to the immobilized antibody was tried. For removal of the mast cells, 3% gelatin gel was used. Similar to other Examples, 3% gelatin gel was ice-cooled to prevent dissolution at room temperature and maintain gel strength. The 3% gelatin gel was fed at 480 sec. Thereafter, buffer A was fed for 480 sec, and the regenerating solution remaining on the chip surface was removed. As a result, after feeding 3% gelatin gel, the reflectance converged to 0% in the mast cells as well, and the mast cells could be completely removed.

### Example 7 Detection of Pseudomonas aeruginosa (NCTC12924) and Regeneration of Sensor Chip

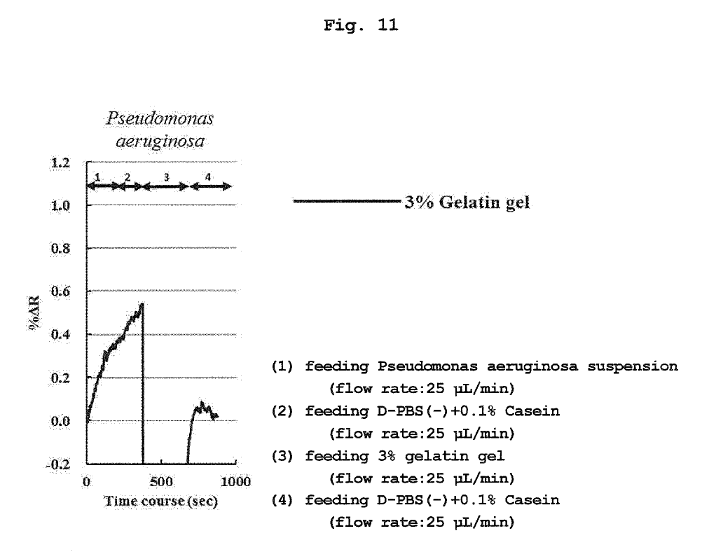

Pseudomonas aeruginosa was cultured in Soybean-casein digest agar (SC agar medium, NIHON PHARMACEUTICAL CO., LTD.). As an antibody to Pseudomonas aeruginosa, a polyclonal antibody purified from immune serum “SEIKEN” I group for Pseudomonas aeruginosa group (DENKA SEIKEN Co., LTD, 213662) was used. Purified antibody was immobilized on a sensor chip surface, and conditioned by feeding buffer C (D-PBS(−) containing 0.1% Casein) at a flow rate of 25 μL/min. The measurement was started with the reflectance at this point as 0%. The Pseudomonas aeruginosa suspended in buffer A was fed to the surface of the sensor chip for 240 sec, and then buffer A was fed for 120 sec. The reflectance at this point was 0.5%, and Pseudomonas aeruginosa was specifically bound to the immobilized antibody on the chip (FIG. 11).

Next, regeneration of the sensor chip, that is, removal of the Pseudomonas aeruginosa bound to the immobilized antibody was tried using 3% gelatin gel. Similar to other Examples, 3% gelatin gel was ice-cooled to prevent dissolution at room temperature and maintain gel strength. The 3% gelatin gel was fed at 240 sec. Thereafter, buffer A was fed for 240 sec, and the regenerating solution remaining on the chip surface was removed. As a result, after feeding 3% gelatin gel, the reflectance converged to 0% in the Pseudomonas aeruginosa as well, and the Pseudomonas aeruginosa could be completely removed.

### INDUSTRIAL APPLICABILITY

Using the removal method of the present invention, a conventionally-disposed carrier immobilized with an antibody, which was used in an immunological detection method of a microorganism or the like, can be reutilized and the economical burden is reduced.

This application is based on patent application Nos. 2015-244569 filed in Japan (filing date: Dec. 15, 2015) and 2016-182211 filed in Japan (filing date: Sep. 16, 2016), the contents of which are incorporated in full herein.

1. A method for removing a microorganism, a cell, a vesicle secreted by the microorganism or the cell, or a virus from an antibody immobilized on a carrier and to which the microorganism, the cell, the vesicle secreted by the microorganism or the cell, or the virus has been bound as an antigen, the method comprising flowing a gel (primary gel) on a surface of the carrier.

2. The method according to claim 1, wherein the primary gel is a polysaccharide gel or a protein gel.

3. The method according to claim 2, wherein the polysaccharide is selected from the group consisting of agarose, agar and carrageenan.

4. The method according to claim 2, wherein the protein is gelatin.

5. The method according to claim 1, wherein the primary gel has a breaking strength of 4-1100 g/cm².

6. The method according to claim 1, wherein the microorganism is Escherichia coli, Streptococcus pneumoniae or Pseudomonas aeruginosa, the cell is an animal cell, and the vesicle secreted by the microorganism or the cell is an exosome.

7. The method according to claim 1, further comprising washing after flowing and flowing again a gel (secondary gel) the same as or different from the primary gel, wherein the primary gel is a mixture further comprising a salt aqueous solution.

8. The method according to claim 7, wherein the primary gel and the secondary gel are each a polysaccharide gel or a protein gel.

9. The method according to claim 8, wherein the polysaccharide is selected from the group consisting of agarose, agar and carrageenan.

10. The method according to claim 8, wherein the protein is gelatin.

11. The method according to claim 7, wherein the primary gel is agarose gel and the secondary gel is gelatin gel.

12. The method according to claim 7, wherein the primary gel and the secondary gel each have a breaking strength of 4-1100 g/cm².

13. The method according to claim 7, wherein the salt aqueous solution is an aqueous ammonium sulfate solution.

14. The method according to claim 7, wherein the microorganism is Escherichia coli, Streptococcus pneumoniae or Pseudomonas aeruginosa, the cell is an animal cell, and the vesicle secreted by the microorganism or the cell is an exosome.

15. A method for immunologically detecting a microorganism, a cell, a vesicle secreted by the microorganism or the cell, or a virus, comprising removing a microorganism, a cell, a vesicle secreted by the microorganism or the cell, or a virus from an antibody immobilized on a carrier and to which the microorganism, the cell, the vesicle secreted by the microorganism or the cell, or the virus has been bound as an antigen, by flowing a gel (primary gel) on a surface of the carrier, contacting a test sample with the carrier, and detecting the microorganism, the cell, the vesicle secreted by the microorganism or the cell, or the virus by an immunological method.

16. The method according to claim 15, further comprising washing after flowing and flowing again a gel (secondary gel) the same as or different from the primary gel, wherein the primary gel is a mixture further comprising a salt aqueous solution.

17. An apparatus for removing a microorganism, a cell, a vesicle secreted by the microorganism or the cell, or a virus from an antibody immobilized on a carrier and to which the microorganism, the cell, the vesicle secreted by the microorganism or the cell, or the virus has been bound as an antigen, the apparatus comprising a mechanism for flowing a gel on a surface of the carrier.
